(19) United States
(12) Patent Application Publication
(10) Pub. No.: US 2019/0210990 A1
(43) Pub. Date: Jul. 11, 2019

(54) METHOD FOR CONVERTING LEVOGLUCOSENONE INTO 4-HYDROXYMETHYL BUTYROLACTONE AND 4-HYDROXYMETHYL BUTENOLIDE WITHOUT USING ANY ORGANIC SOLVENT AND CATALYST

(71) Applicant: INSTITUT DES SCIENCES ET INDUSTRIES DU VIVANT ET DE L'ENVIRONNEMENT - AGROPARISTECH, Paris (FR)

(72) Inventors: Florent ALLAIS, BOUY (FR); Guillaume BONNEAU, REIMS (FR); Aurélien PERU, REIMS (FR); Amandine FLOURAT, REIMS (FR)

(73) Assignee: INSTITUT DES SCIENCES ET INDUSTRIES DU VIVANT ET DE L'ENVIRONNEMENT - AGROPARISTECH, Paris (FR)

(21) Appl. No.: 16/315,143

(22) PCT Filed: Jul. 6, 2017
(86) PCT No.: PCT/FR2017/051848

§ 371 Date: Jan. 7, 2019

(30) Foreign Application Priority Data
Jul. 7, 2016 (FR) 1656565

(51) Int. Cl. C07D 307/58; C07D 307/32

(52) U.S. Cl. CPC C07D 307/58; C07D 307/32

(57) ABSTRACT

The invention relates to a method for converting levoglucosenone into 4-hydroxymethyl butyrolactone and 4-hydroxymethyl butenolide without using any solvent or catalyst, wherein the oxidation and hydrolysis of the levoglucosenone or hydrogenated levoglucosenone are carried out in a single step using an aqueous solution of H₂O₂.

The present invention relates to the transformation of levoglucosenone (LGO) into chemical intermediates used in the food and pharmaceutical industries. The chemical intermediates obtained from levoglucosenone are 4-hydroxymethylbutyrolactone (2H-HBO) and 4-hydroxymethylbutenolide (HBO).

Production processes of chemical compounds of interest from biomass have been developed over the last decades in order to solve the problem of fossil fuel dependency. In particular, lignocellulosic biomass has become one of the green sources of carbon compounds. Levoglucosenone, of formula (Ia):

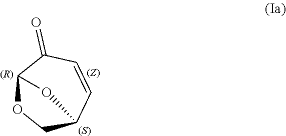

is one of the most interesting products that can be obtained from biomass, notably by a technology of cellulose flash pyrolysis.

Levoglucosenone is commonly used as a starting material for the synthesis of various chemical compounds of interest, in particular 4-hydroxymethylbutenolide (HBO), of formula (IIa) and 4-hydroxymethylbutyrolactone (2H-HBO), of formula (IIb).

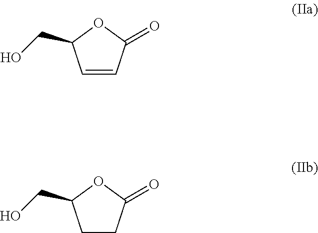 [SMILES: O=C1C=C[C@@H](CO)O1 ; O=C1CC[C@@H](CO)O1]

These compounds are of special interest as they are asymmetrical (chiral) chemical intermediates with high added value which are frequently used in the food industry, to generate fragrances and flavours, or the pharmaceutical industry, for the production of active ingredients in drugs, taking advantage of their lactone ring and their chiral centre.

The transformation of levoglucosenone into 4-hydroxymethylbutenolide is conventionally performed in two consecutive steps, the first one consisting in a Bayer-Villiger oxidation reaction to form a formate intermediate, followed by an acidic hydrolysis step to form 4-hydroxymethylbutenolide. In order to obtain 4-hydroxymethylbutyrolactone, levoglucosenone is firstly submitted to a step of catalytic hydrogenation, so as to obtain dihydrolevoglucosenone, which then undergoes the transformation steps described above with reference to levoglucosenone.

### PRIOR ART

Transformation processes of levoglucosenone into 4-hydroxymethylbutyrolactone and 4-hydroxymethylbutenolide are known in the prior art.

The transformation of levoglucosenone into 4-hydroxymethylbutenolide is conventionally performed in two consecutive steps, the first one consisting in a Bayer-Villiger oxidation reaction to form a formate intermediate, followed by an acidic hydrolysis step to form 4-hydroxymethylbutenolide.

In order to obtain 4-hydroxymethylbutyrolactone, levoglucosenone is firstly submitted to a step of catalytic hydrogenation, so as to obtain dihydrolevoglucosenone, which then undergoes the transformation steps described above with reference to levoglucosenone.

Several methods for implementing the oxidation reaction of levoglucosenone or dihydrolevoglucosenone have been proposed in the prior art.

By way of example, document U.S. Pat. No. 4,994,585, describing the oxidation of levoglucosenone by a peracid such as m-chloroperbenzoic acid or peracetic acid, in an organic solvent; or document U.S. Pat. No. 5,112,994, describing a method for the synthesis of 4-hydroxymethylbutyrolactone from dihydrolevoglucosenone, according to which the oxidation reaction is also performed by a peracid in an organic solvent, may be cited. However, these reactions must be conducted during long periods of time, from one to two days, in order to achieve high yields.

Alternatively, the publication of Paris et al., Green Chemistry, 2013, 15, 2101-2109, proposes to perform the oxidation reaction of levoglucosenone with metallic catalysts, such as aluminium or tin zeolites, in the presence of an oxidising agent. However, such methods may also be time-consuming, for some of the proposed catalysts, if satisfactory conversion rates of levoglucosenone are to be obtained. Aluminium zeolites allow high conversion rates to be obtained in four hours. However, this duration does not account for the time necessary for the preparation of the catalyst. Furthermore, the catalyst is potentially toxic.

In addition, it has been proposed in the prior art, with the aim of being more environmental-friendly by using less toxic compounds, to perform the oxidation of cyclic ketones through a Bayer-Villiger reaction by means of a lipase immobilized on a particular medium of grafted porous silica as a catalyst. In particular, such a process is described in Drozdz et al., in Applied Catalysis A: General, 2013, 467, 163-170. According to this document, the process is satisfactory, in terms of conversion rate of the cyclic ketone and of reaction time, for ketones with high ring strain such as cyclobutanone or cyclopentanone, but only for this type of cyclic ketones. On the contrary, for cyclic ketones with low ring strain, such as cyclohexanone or cycloheptanone, such an oxidation reaction turns out to be inefficient, with low conversion rates and/or very long reaction times. Such a difference of reactivity according to ring strain, related in particular to the number of members in the cycle, is well known to the person skilled in the art, whose general knowledge in this field is illustrated, for example, in Wiberg, Angew. Chem. Int. Ed. Engl., 1986, 25, 312-322.

WO2015/165957 describes a process for transforming levoglucosenone into 4-hydroxymethylbutyrolactone and 4-hydroxymethylbutenolide, including a step of oxidation of levoglucosenone or dihydrolevoglucosenone obtained by hydrogenation of levoglucosenone, by contacting a solution of levoglucosenone and a solvent, with a lipase and an acyl-giving compound, followed by a step of dihydrolevoglucosenone hydrolysis in the presence of an oxidizing agent, this latter oxidation step being of the obtained reaction solution, and, as the case may be, by a hydrogenation step of the compound obtained from this hydrolysis step.

### DRAWBACKS OF THE PRIOR ART

The solutions of the prior art all involve a two-step synthesis that may take up to two days. The solutions of the prior art always use a solvent and a catalyst. The person skilled in the art is thus systematically forced to transform levoglucosenone into 4-hydroxymethylbutyrolactone and 4-hydroxymethylbutenolide in two steps, in the presence of a solvent and a catalyst. In the state of the art, this catalyst is either chemical, for example zeolites, or biological, for example a lipase-type enzyme. However, traces of these solvents and catalysts remain in the finished product, even after extensive purification. The desired purity of the obtained product is thus not achieved. These traces have some toxicity, in particular tin-based catalysts, which eventually may cause health problems for the consumer. Likewise, these trace compounds may alter the performance of the syntheses of molecules of which HBO and 2H-HBO are synthesis intermediates.

### SOLUTION PROVIDED BY THE INVENTION

Unexpectedly, the authors of the present invention have found that the transformation of levoglucosenone into 4-hydroxymethylbutyrolactone and 4-hydroxymethylbutenolide could be achieved in a single step, without any organic solvent or catalyst, with high yields. Consequently, the solution of the present invention is simple, non toxic, more environmental-friendly and provides high yields. The yields obtained with the process of this invention are identical or even higher than those obtained with the prior art processes using chemical or biological catalysts. Moreover, the process according to this invention can be conducted as a continuous flow production. The absence of organic solvent and catalyst in the process of this invention allows products of a higher level of purity than those obtained with the prior art processes to be obtained. This is of particular interest as these products are intermediates used in the food industry, especially in foods for humans. This limits the impact on both the environment and health. This also limits the impact of impurities on the subsequent syntheses by comparison to the methods of the prior art. Moreover, the process according to this invention is particularly cost-efficient.

### DETAILED DESCRIPTION OF THE INVENTION

In its general meaning, the present invention intends to remedy the drawbacks of the prior art with a process of transforming levoglucosenone into a compound of general formula (II):

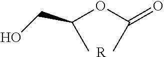

in which R represents —CH═CH— or —CH₂—CH₂—, comprising the following consecutive steps:

a) as the case may be, to obtain a compound of general formula (II) wherein R represents —CH₂—CH₂—, hydrogenation of levoglucosenone to form dihydrolevoglucosenone,

b) oxidation of levoglucosenone or of dihydrolevoglucosenone obtained in step a),

c) hydrolysis of the reaction mixture obtained in step b),

d) as the case might be, to obtain a compound of general formula (II) wherein R represents —CH₂—CH₂—, if step a) was not performed, hydrogenation of compound obtained in step c),

characterised in that the oxidation of step b) and the hydrolysis of step c) are performed in a single step in the presence of an aqueous solution of H₂O₂, without involving any organic solvent or catalyst.

The process according to the invention allows the Bayer-Villiger oxidation and the hydrolysis of the formate intermediate to take place in a single reaction. Unlike the solutions of the prior art, the hydrolysis does not require the implementation of a second reaction step using acids and organic solvents. Unlike the solutions of the prior art using, in order to conduct the Bayer-Villiger reaction on the levoglucosenone or dihydrolevoglucosenone, H₂0₂, a catalyst (chemical such as zeolites or biological such as a lipase) and an organic solvent (such as 1,4-dioxane or ethyl acetate), the process of this invention uses only H₂0₂. H₂0₂ is in the form of an aqueous solution of H₂0₂, H₂0₂-urea or 2Na₂CO₃.3H₂O₂. Unexpectedly, in the process of this invention, the hydrolysis is performed at the same time as the oxidation.

The process of this invention uses an aqueous solution of H₂0₂, water, perfectly non toxic, acting as a solvent.

The reaction according to the invention thus excludes any type of organic and inorganic solvent, except water.

In the present invention, “organic solvent” means any carbon-containing solvent. Organic solvents are classified into three families:
1) hydrocarbon solvents comprising aliphatic solvents such as alkanes and alkenes, aromatic solvents such as benzene, toluene and xylene;
2) oxygen-containing solvents such as alcohols (ex: ethanol, methanol), ketones (ex: acetic acid), esters (ex: ethyl acetate), ethers (ex: ether, glycol ether) and other solvents (ex: DMF, DMSO and HMPT);
3) halogen-containing solvents such as halogenated hydrocarbons, i.e. fluorinated, chlorinated, brominated or iodinated (ex: perchloroethylene, trichloroethylene, dichloromethane, chloroform, tetrachloromethane).

In the present invention, “inorganic solvent” means any solvent containing no carbon.

Besides water, aqueous solutions containing additives (notably surfactants), concentrated sulphuric acid, ammonia are classical inorganic solvents.

The hydrogenation reaction can be performed according to any method known to the person skilled in the art, particularly catalytic hydrogenation, for example in the presence of palladium on carbon and hydrogen.

The reaction time in the presence of an aqueous solution of H₂0₂is advantageously comprised between 2 and 53 hours.

In one embodiment, the reaction temperature is from −78° C. to 50° C., preferably from −78° C. to 40° C., or even from −78° C. to 20° C., during the addition of the aqueous solution of H₂O₂ over a duration of 5 minutes to 8 hours so as to control the exothermicity of the addition, after which the reaction temperature is comprised between 2° C. and 150° C. for a duration of at least 4 hours, notably from 4 to 48 hours, preferably from 8 to 48 hours once the addition of the H₂0₂ solution has been completed.

In a preferred embodiment, the reaction temperature is 0° C. during the addition of the aqueous solution of H₂O₂ over a duration of 3.5 hours or even 4 hours, after which the reaction temperature is 50° C. for a duration of 20 hours, preferably of 8 hours once the addition of the H₂0₂ solution has been completed.

The concentration of the aqueous solution of H₂0₂ is comprised between 20 and 60%.

Preferably, the concentration of the aqueous solution of H₂0₂ is from 30% to 50%.

The number of H₂0₂ equivalents is comprised between 0.8 and 10 based on the number of moles of starting levoglucosenone.

In one embodiment, the number of H₂0₂ equivalents is 1.2 based on the number of moles of starting levoglucosenone. In a preferred embodiment, the number of equivalents is 1.

The larger the quantity of H₂0₂, the more the reaction is rapid.

A quantity of 1.2 equivalents is particularly advantageous. In a still more advantageous embodiment, this equivalent quantity is 1. These quantities allow a high efficiency to be obtained, with a good conversion rate/yield while minimising the quantity of reagent. This quantity is more economical. This low quantity of H₂0₂ also avoids a quenching step of the excess H₂0₂. This quantity of 1.2 or 1 equivalent, thus represents an optimal balance between conversion rate/yield, reaction time, implementation and cost.

Advantageously, the excess oxidant present in the reaction mixture can be quenched by heating at a high temperature; this «high temperature» is comprised between 70° C. and 150° C. Preferably, the temperature applied is comprised between 80° C. and 100° C., and still more preferably is 90° C.

Advantageously, the process requires no organic solvents (other than water) and no catalysts (either metallic or biological).

Thus, the process according to this invention allows synthesis of preparations of HBO and 2H-HBO containing no trace metals from metallic catalysts, no trace proteins from biological catalysts and no trace of organic solvent. This preparation is of a purity never attained by the methods of the prior art.

Consequently, the present invention concerns a preparation of HBO or 2H-HBO containing no traces of metal, organic solvent or protein.

The preparations of the invention are thus different from those obtained by the methods of the prior art.

Moreover, the HBO and 2H-HBO thus available in preparations devoid of toxic contaminants, are particularly appropriate for the preparation of derived molecules to be used in the food, cosmetic or pharmaceutical industries.

The present invention thus also concerns the use of preparations of HBO and 2H-HBO according to the invention in applications aimed at foods, cosmetics or therapeutics.

The present invention will be better understood in the light of non exhaustive examples of embodiment.

### BRIEF DESCRIPTION OF THE FIGURES

FIG. 1 shows the diagram of the synthesis according to the invention. (A) Simplified representation, (B) Representation of the reaction at the molecule structural level.

FIG. 2 shows the percentage of HBO formed over time based on the quantity of H₂O₂ in the reaction mixture.

FIG. 3 represents the monitoring by HPLC of the formation of E-caprolactone according to Example 5.

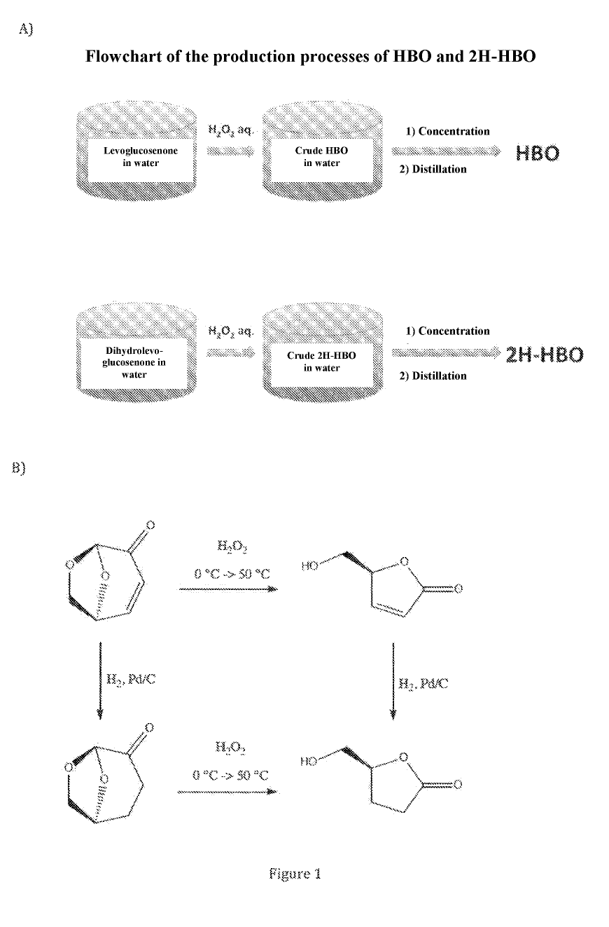

### EXAMPLES

### Example 1: Synthesis of 4-hydroxymethylbutenolide (HBO)

### A—Reaction Involving 1.2 Equivalents of H₂0₂ Based on the Number of Starting Moles of LGO

A 30% aqueous solution of hydrogen peroxide H₂O₂ (9.78 M, 25 mL, 0.245 mol, 1.2 equivalent based on levoglucosenone) was added drop wise with a syringe pump (8 mL/h) under nitrogen over 3.5 hours to the levoglucosenone (25.7 g) under stirring in a water bath with ice. The reaction mixture was heated at 50° C. during 20 h under stirring.

The excess oxidant was quenched at low temperature (in a water bath containing ice) while adding small quantities of sodium sulphite (3 g, 23.8 mmol) under stirring. This is controlled by peroxide test strips.

Sodium sulphite de sodium may be replaced by sodium bisulphite, sodium metabisulphite or a catalase.

Advantageously, the excess oxidant may alternatively be quenched by heating at high temperatures, for example about 90° C. By so doing, the use of «quenchers» is avoided, as well as their subsequent removal.

### Extraction—Purification

Purification may carried out as follows: 150 mL toluene was added to the reaction mixture and concentrated to dryness. The residue was taken up in acetone (25 mL) and filtered on Celite, then rinsed with 10 mL acetone. The filtrate (direct injection of fluid) was purified by silica-gel chromatography (220 g, 30 micron), eluted with 50 to 100% ethyl acetate in cyclohexane to give 16.8 g of pure HBO (72%) as a colorless oil which crystallised

### B—Reaction Involving 1 Equivalent of H₂0₂ Based on the Number of Starting Moles of LGO

A 30% aqueous solution of hydrogen peroxide H₂O₂ (9.78 M, 0.81 mL, 7.92 mol, 1 equivalent based on levoglucosenone) was added drop wise with a syringe pump over 4 hours to a levoglucosenone solution (1 kg, 7.93 mol) in 1 L water under stirring in a water bath with ice. The reaction mixture was heated at 50° C. during 6 h under stirring. The absence of residual peroxide was controlled by peroxide test strips.

### Extraction—Purification

Purification may be conducted by the method described in A. In this example, the reaction mixture was concentrated under vacuum, then purified by distillation to give 647 g of pure HBO (71%) as a colourless oil that crystallised.

### Example 2: Synthesis of 4-hydroxymethylbutyrolactone (2H-HBO)

### Pathway 1: Catalytic Hydrogenation of the HBO Obtained in Example 1

The 4-hydroxymethylbutenolide (HBO) (IIa) obtained in Example 1 was subjected to catalytic hydrogenation in the following manner.

Pd/C (10% w/w, 140 mg) was added to a solution of 4-hydroxymethylbutenolide (1.4 g, 12.3 mmol) in ethyl acetate (15 mL) at room temperature. Under stirring, the suspension was degassed 3 times under vacuum/nitrogen. The suspension was then hydrogenated under hydrogen atmosphere at room temperature during 4 hours. The crude mixture was filtered on Celite and the filtrate concentrated to dryness. The crude product was purified by distillation or silica-gel chromatography (elution with a gradient from 75 to 100% ethyl acetate in cyclohexane), to obtain pure 4-hydroxymethylbutyrolactone (2H-HBO) of formula (IIb) (1.19 g, 82%).

¹H NMR (CDC1₃, 300 MHz): δ¹H 2.20 (m, 2H) , 2.61 (m, 3H) , 3.66 (dd, 1H, J=12.6 and 4.5 Hz), 3.92 (dd, 1H, J=12.6 and 2.7 Hz), 4.64 (m, 1H)

¹³C NMR (CDC1₃, 75 MHz): δ_{c}, 23.1 (t), 28.7 (t), 64.1 (t), 80.8 (d), 177.7 (s)

### Pathway 2: Hydrogenation of Levoglucosenone Followed by Synthesis

### Catalytic Hydrogenation of Levoglucosenone

Pd/C (10% w/w, 500 mg) was added to a solution of (-)-levoglucosenone LGO (5 g, 39.7 mmol) in ethyl acetate (50 mL) at room temperature. Under stirring, the suspension was degassed 3 times under vacuum/nitrogen. The suspension was then hydrogenated in a hydrogen atmosphere at room temperature until complete consumption of the starting product, i.e. For about 4 h. The crude mixture was filtered on Celite and the filtrate was concentrated to dryness. The crude product was purified by distillation or silica-gel chromatography (elution with a gradient from 10 to 60% ethyl acetate in cyclohexane), to obtain pure dihydrolevoglucosenone of formula (Ib) (2H-LGO) (colourless oil, 4.4 g, 87%).

¹H NMR (CDC1₃, 300 MHz): δ¹H 2.02 (m, 1H) , 2.34 (m, 2H), 2.62 (m, 1H), 4.00 (m, 2H), 4.70 (m, 1H), 5.10 (s, 1).

¹³C NMR (CDC1₃, 75 MHz): δ_{c}, 29.9 (t), 31.1 (t), 67.5 (t), 73.1 (d), 101.5 (d), 200.3 (s)

### Bayer-Villiger Reaction

Option 1: A 30% aqueous solution of hydrogen peroxide H₂O₂ (9.78 M, 25 mL, 0.245 mol, 1.2 equivalent based on dihydrolevoglucosenone) was added drop wise with a syringe pump (8 mL/h) under nitrogen over 3.5 hours to the dihydrolevoglucosenone (26.1 g) under stirring in a water bath with ice. The reaction mixture was heated at 50° C. during 20 h under stirring. The excess oxidant was quenched at low temperature (in an iced water bath) while adding small quantities of sodium sulphite (3 g, 23.8 mmol) under stirring. This was controlled using peroxide test strips.

### Extraction—Purification

150 mL toluene were added to the reaction mixture, which was concentrated to dryness. The residue was taken up in acetone (25 mL) and filtered on Celite, then rinsed with 10 mL acetone. The filtrate (direct injection of fluid) was purified by silica-gel chromatography (220 g, 30 micron), eluted with 50 to 100% ethyl acetate in cyclohexane to give 16.8 g of pure 2H-HBO (72%) as a colourless oil which crystallised.

The extraction-purification step of 2H-HBO may be carried out by concentrating the reaction mixture under vacuum, then by purifying it by distillation, as described above for HBO.

Option 2: A 30% aqueous solution of hydrogen peroxide H₂O₂ (9.78 M, 0.81 mL, 7.92 mol, 1 equivalent based on dihydrolevoglucosenone) was added drop wise with a syringe pump over 4 hours under nitrogen to a dihydrolevoglucosenone solution (1.02 kg, 7.93 mol) in 1 L water under stirring in a water bath with ice. The reaction mixture was then heated at 50° C. during 6 h under stirring. The absence of residual peroxide was controlled with peroxide test strips.

### Extraction—Purification

The extraction-purification step of 2H-HBO may be carried out by concentrating the reaction mixture under vacuum, then by purifying it by distillation, as described above for HBO.

Both the above paths of synthesis 1 and 2 allow to obtain, in a regioselective manner and with high yields, 4-hydroxymethylbutyrolactone (IIb, 2H-HBO), the structure of which being confirmed by proton and carbon NMR.

### Example 3: Monitoring the Transformation of LGO into HBO

### Kinetic Monitoring of the Reaction by HPLC

### HPLC Protocol

A sample of 2.5 μL reaction medium was diluted in 1.5 mL acetonitrile. The progress of the reaction was followed by HPLC and the formation of HBO was monitored at 220 nm.

The analyses were performed on a C18 Thermo Scientific® Syncronis® aQ column (250×4,6 mm, 5 μm) under the following conditions: injected volume 10 μL; oven temperature 30° C.; flux: 0.8 mL·min⁻¹; elution method: isocratic 85/15 water/acetonitrile from 0 to 5 min, 5 to 10 min 85/15 to 90/10 water/acetonitrile gradient, 10 to 15 min isocratic 90/10 water/acetonitrile, 15 to 20 min gradient of 90/10 to 85/15 water/acetonitrile; recording of the spectre at 220 nm.

The excess hydrogen peroxide at the end of the reaction was quenched with a small amount of sodium sulphite (Na₂SO₃+H₂O₂−>Na₂SO₄+H₂O) at room temperature. The reaction was stirred for 5 min.

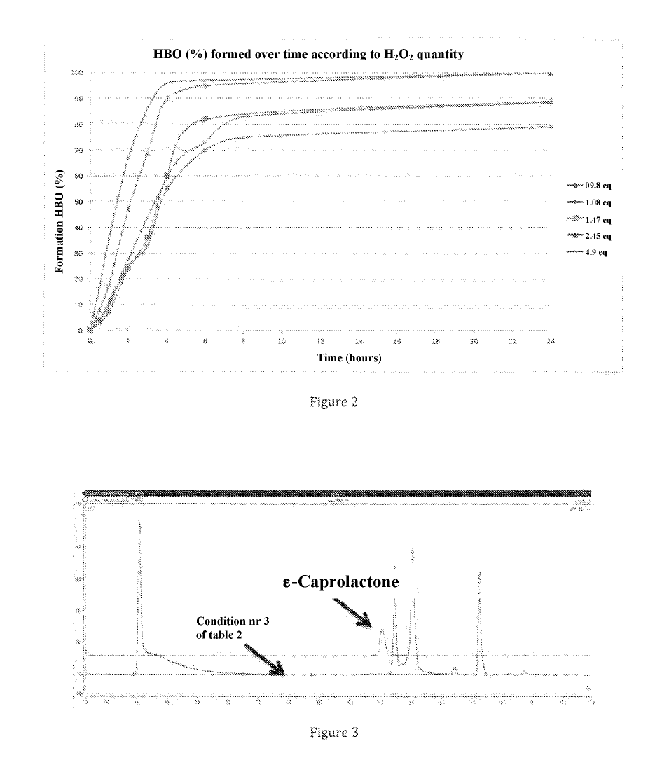

2.5 μL samples were taken from the reaction mixture at different time intervals and analysed by HPLC according the above-described protocol. Different quantities if H₂O₂ were compared (FIG. 2). The tested H₂0₂ quantities according to the synthesis process of the invention were 0.98, 1.98, 1.47, 2.45 and 4.9 equivalents of H₂0₂ based on the initial quantity of starting product. The more the H₂0₂ quantity is large, the larger is the quantity of HBO formed (FIG. 2).

### Example 4: Comparative Study of the Process of the Invention and of a Prior Art Process

### HBO was Synthesised

according to the process of the invention as described above
and according to two methods of the prior art
   by the lipase pathway as described in A. L. Flourat, A. A. M. Peru, A. R. S. Teixeira, F. Brunissen, F. Allais, Green Chem., 2015, 17, 404-412 and in the patent application WO2015/165957,
   by the zeolite pathway as described in C. Paris, M.

Molier, A. Corma, Green Chem., 2013, 15, 2101-2109.

### Synthesis of HBO by the Lipase Pathway

### Oxidation

In a reactor, a 30% aqueous solution of hydrogen peroxide H₂O₂ (2.57 mmol, 0.26 mL, 1.2 eq. based on LGO) was added in a single portion to a suspension of LGO (270 mg, 2.14 mmol) and lipase CaL-B (Novozym® 435, 75 mg, 315 U/mmol LGO) in ethyl acetate (3 mL) under stirring at room temperature, in an incubator with planar stirring. For this example as well as all the following examples, 1 g of Novozym® 435 corresponds to 9000 units of lipase CaL-B (activity measured after the enzyme was mixed with ethyl acetate). The reaction mixture was stirred at 40° C. for 4 h then evaporated to dryness.

### Acid Hydrolysis

Concentrated hydrochloric acid (5 mmol, 0.4 mL) was added to a solution of this crude mixture in methanol (5 mL), at room temperature. The reaction mixture was heated under stirring during 8 to 16 hours so as to convert the formate (IIIa) into the corresponding alcohol (IIa). The reaction mixture was evaporated to dryness on silica gel. The crude product was purified by silica gel chromatography (elution with 75 to 100% ethyl acetate in cyclohexane) to obtain pure 4-hydroxymethylbutenolide (IIa) (175 mg, 72%).

¹H NMR (CDC1₃, 300 MHz): d 7.53 (dd, J=1.5 and 5.7 Hz, 1 H), 6.2 (dd, J=1.5 and 5.7 Hz, 1 H), 5.17 (m, 1 H), 4.0 (d, J=3.6 and 12.0 Hz, 1 H), 3.80 (dd, J=3.6 and 12.0 Hz, 1 H), 3.25 (s, 1 H)

¹³C NMR (CDC1₃, 75 MHz): d 173.5 (s), 154.0 (d), 122.8 (d), 84.3 (d), 62.2 (t)

### Synthesis of HBO by the Zeolite Pathway

### Oxidation

In a reactor, levoglucosenone (0.33 mmol, 42 mg) was dissolved in 1 mL 1,4-dioxane and a solution of hydrogen peroxide (35% in water, 0.5 mmol) was added to the solution. This mixture was contacted the the zeolites (levoglucosenone/metal of the zeolite=20) and the reaction was maintained at 100° C. for 4 hours. The solution was then filtered.

### Acid Hydrolysis

The previously filtered solution was left in contact with 140 mg of acidic resin Amberlyst-15 at room temperature for 6 hours.

### Comparison of the Three Techniques

The comparison of the three methods of synthesis (Table 1) shows that synthesis by the process of this invention is carried out in a single step, whereas synthesis requires two steps in both of the other methods.

The yield of the process of the invention is 72%, which is higher than the yield obtained through the lipase pathway. The yield of the zeolite pathway is 89%, but this is not isolated, unlike the yield of the process according to this invention. Consequently, the isolated yield of the process according to the invention is superior to the yields of the prior art methods.

The process according to the invention requires only one reagent, H₂0₂, whereas the lipase and zeolite pathways require a number of reagents. The process according to the invention is thus more advantageous and cost-efficient than the processes of the prior art.

The process according to the invention does not use large quantities of organic solvents or metallic catalysts, unlike the methods of the prior art. Consequently, it is more environmental-friendly. Also, the obtained product has a higher purity level and, ipso facto, contains no traces of solvent or metallic catalyst. This is particularly convenient as HBO and 2H-HBO are both intermediates used for the synthesis of flavours in the food and pharmaceutical industries. Consequently, this limits the potential side effects, such as the risks of allergy and toxicity, for the end consumer.

TABLE 1
Comparison between the synthesis process
according to the invention and two methods of the prior art

| | H2O2 only | Lipase pathway | Zeolite pathway |
|---|---|---|---|
| Yield isolated | 72% | 67% | 89% (not isolated) |
| Reaction time | 23.5 hours | 1st step: 2 hours | Pre-step: 4 hours |
| | | 2nd step: 6-8 hours | 2nd step: 6 hours |
| Temperature | 3.5 hours: 0° C. | 40° C. | 100° C. |
| | 20 hours: 50° C. | | |
| Number of | 1 | 2 | 2 |
| steps | | | |
| Quantity of | 1.2 equivalent | CAL-B (catalytic | 1.5 equivalent |
| reagents | H2O2 (30%) | quantity) | H2O2 (35%) |
| | | Solid pad (20 | Sn-Beta catalyst |
| | | mg/mL) | Amberlyst 15-H |
| | | 1.2 equivalent H2O2 | |
| | | (50%) | |
| | | Amberlyst 15-H | |
| Solvent | No organic | Ethyl acetate (C = | 1,4-dioxane (C = |
| | solvent | 0.5-1M) | 0.33M) |

### Example 5: The Process According to the Invention Does Not Work With Any Ketone

The process according to the invention is specific for levoglucosenone. Any ketone may not be used as a substrate.

The process according to the invention has been tested with a conventional ketone, namely cyclohexane. Levoglucosenone and cyclohexanone are cyclic ketones with 6-member rings.

Different quantities of 30% aqueous solution of hydrogen peroxide H₂0₂ were tested, with or without solvents and also with an enzyme catalyst such as described in WO2015/165957.

A 30% aqueous solution of hydrogen peroxide H₂0₂ (1.2 equivalent based on cyclohexanone, 0.59 mL, 5.8 mmol or 2.5 equivalent based on cyclohexanone, 1.2 mL, 12.1 mmol) was added in one go to the cyclohexanone solution (0.5 mL, 4.84 mmol) under stirring and cooled in a water bath with ice with (condition 2) or without (condition 1, corresponding to the conditions of application WO2015/165957) ethanol (2 mL). The mixture was left to cool in a water bath with ice for one hour. The reaction mixture was then heated at 50° C. during 18 h under stirring.

For condition 2, an aliquot (0.5 ml) was taken and maintained under stirring with sodium sulphite in a water bath with ice. This mixture was then diluted with ethanol, filtered and concentrated to dryness.

The consumption of starting substrate was monitored by TLC (thin layer chromatography) (elution 7/3 cyclohexane/ethyl acetate, then revealed with a vanilline solution).

The formation of E-caprolactone was monitored by HPLC (FIG. 3), high-performance liquid chromatography,

(Thermofisher Ultimate 3000 equipped with a DAD detector) using a C18 column (Syncronis aQ 250×4.6 mm, 5 μm). 50 μL samples were added to 0.5 mL acetonitrile. 30 μL of these mixtures were injected in the HPLC device at a temperature of 30° C. with a flux of 0.8 mL·min⁻¹. The elution method (water-acetonitrile) was as follows: 0-5 min 90/10, 5-10 min from 90/10 to 0/100, 10-20 min 0/100, 20-27 min from 0/100 to 90/10. UV detector at 254 and 280 nm.

TABLE 2
The process according to the invention does not
work with other ketones.

| | H202 | | |
|---|---|---|---|
| Conditions | 30% eq | solvent | Comment |
| 1 (process | 1.2 | — | No reaction |
| according to | | | |
| the invention) | | | |
| 2 | 1.2 | EtOH (2.4M) | No reaction |
| 3 | 2.5 | — | Partial disappearance of the |
| | | | starting product |
| | | | New product observed with |
| | | | TLC and analysed with ¹H NMR |
| | | | and HPLC: no presence of |
| | | | E-caprolactone |
| 4 | 2.5 | EtOH (2.4M) | No reaction |
| 5 (W02015/ | 1.1 | Ethyl | 40% of starting product |
| 165957)* | | acetate + | consumed at 24 hours (GCMS) |
| | | N435 | |
| | | (lipase) | |

*(Conditions of the patent application WO2015/165957: the reaction mixture comprised 25 mg N435 (525 U/mmol), 0.5 mmol cyclohexanone and 0.6 mmol H₂O₂ and ethyl acetate to a final volume of 1 mL. The reaction was carried out for 48 h at 40° C. under stirring at 400 rpm.

The transformation process which works very well on LGO does not work at all on cyclohexanone (FIG. 3). FIG. 3 shows that E-caprolactone usually has a retention time of 11 min. Under the conditions nr 3 of table 2, E-caprolactone is not formed. Consequently, the process according to this invention does not work on any ketone and is levoglucosenone-specific.

1. Process for transforming levoglucosenone into a compound of general formula (II): 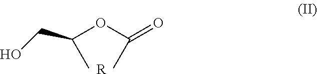
wherein R represents —CH═CH— or —CH₂—CH₂—, comprising the following consecutive steps:
a) as the case may be, to obtain a compound of general formula (II) wherein R represents —CH₂—CH₂—, hydrogenation of levoglucosenone to form dihydrolevoglucosenone,
b) oxidation of levoglucosenone or of dihydrolevoglucosenone obtained in step a),
c) hydrolysis of the reaction mixture obtained in step b),
d) as the case may be, to obtain a compound of general formula (II) wherein R represents —CH₂—CH₂—, if step a) was not performed, hydrogenation of the compound obtained in step c), characterized in that oxidation in step b) and hydrolysis in step c) are performed in a single step in the presence of an aqueous solution of H₂0₂.

2. The process according to claim 1 characterised in that the reaction time in the presence of an aqueous solution of H₂0₂ is comprised between 2 and 53 hours.

3. The process according to any of claim 1 or 2 characterised in that the reaction temperature is −78° C. to 50° C. during the addition of the aqueous solution of H₂O₂ over a duration of 5 minutes to 8 hours, after which the reaction temperature is comprised between 20° C. and 150° C. for a duration of 4 hours to 48 hours once the addition of the H₂0₂ solution is complete.

4. Process according to any of claims 1 to 3 characterised in that the reaction temperature is 0° C. during the addition of the aqueous solution of H₂O₂, over a duration of
4 hours, the reaction temperature being maintained at 0° C. for 8 hours once the addition of the aqueous solution of H₂O₂ is completed, then at 50° C. during 8 hours.

5. Process according to any one of the claims from 1 to 4, characterised in that the concentration of the H₂O₂ aqueous solution is comprised between 20 to 60%.

6. Process according to any one of the claims from 1 to 5, characterised in that the concentration of the H₂O₂ aqueous solution is 30% or 50%.

7. Process according to any one of claims 1 to 6, characterised in that the number of H₂0₂ equivalents is comprised between 0.8 and 10 based on the number of moles of starting levoglucosenone.

8. Process according claim 7, characterized in that the number of H₂0₂ equivalents is 1 based on the number of moles of starting levoglucosenone.

9. Process according to any one of claims 1 to 8 characterised in that it requires no organic solvent other than water and no catalyst.

10. Process according to any one of claims 1 to 9, further comprising a step of quenching the excess oxidant by heating.

11. Preparation of HBO or 2H-HBO, characterised in that it contains no trace of metal, no organic solvent and no protein.

12. Use of the preparation according to claim 11 in applications for the food, cosmetic and pharmaceutical industries.
